(19) United States
(12) Patent Application Publication
(10) Pub. No.: US 2024/0299375 A1
(43) Pub. Date: Sep. 12, 2024

(54) USE OF MEVIDALEN AND OTHER D1 POSITIVE ALLOSTERIC MODULATORS IN THE TREATMENT OF HALLUCINATIONS AND DEMENTIA-RELATED PSYCHOSIS

(71) Applicant: Eli Lilly and Company, Indianapolis, IN (US)

(72) Inventors: Kevin Michael Biglan, Indianapolis, IN (US); Kjell Anders Ivan Svensson, Indianapolis, IN (US)

(21) Appl. No.: 18/549,546

(22) PCT Filed: Mar. 8, 2022
(86) PCT No.: PCT/US2022/019308

§ 371 Date: Sep. 7, 2023

Related U.S. Application Data
(60) Provisional application No. 63/157,966, filed on Mar. 8, 2021.

(51) Int. Cl. A61K 31/472; A61K 9/00; A61K 45/06; A61P 25/16; A61P 25/18; A61P 25/28

(52) U.S. Cl. CPC A61K 31/472; A61K 9/0053; A61K 45/06; A61P 25/16; A61P 25/18; A61P 25/28

(57) ABSTRACT

The present invention relates to methods of treating and dosing regimens using Mevidalen, also described as 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone, and/or pharmaceutical compositions thereof, for treatment of hallucinations and/or psychosis, including dementia-related psychosis.

The present application claims the benefit of priority to U.S. Provisional Patent Application No. 63/157,966, filed on Mar. 8, 2021, the entire disclosure of which is incorporated by reference herein.

The present disclosure provides methods of using mevidalen, also described as 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone, and/or co-crystals and pharmaceutical compositions thereof, and/or other dopamine D1 positive allosteric modulators, for the treatment of hallucinations and/or psychosis.

Several neuropsychiatric disorders are associated with development of hallucinations and psychosis. Psychosis is a common pathology of dementia and becomes more frequent with disease progression. Patients likely to have hallucination and psychosis include those having schizophrenia, bipolar disorders, and depression, and neurodegenerative disorders such as Alzheimer's Disease, Lewy Body Dementia, Parkinson's Disease (PD) and Huntington's Disease, Vascular dementia (VaD), and frontotemporal dementia (FTD). Among patients with PD, psychosis occurs in up to 60% of patients over the course of their disease. Psychosis is common across dementia types with a prevalence of 20% to 70%. The presence of neuropsychiatric signs and symptoms in neurodegenerative diseases is predictive of increased caregiver burden, decreased quality of life, earlier progression to nursing home care, severe dementia, and death. There is a close relationship between the clinical manifestations of dementia-related psychosis (DRP) and morbidity/mortality in many neurodegenerative diseases.

Currently, Neuplazid is the only pharmacologic treatment approved for dementia-related psychosis. Atypical antipsychotics are frequently used to treat these disorders despite significant safety concerns. (Pimavanserin: Potential Treatment For Dementia-Related Psychosis, J. Cummings ET AL., The Journal of Prevention of Alzheimer's Disease—JPAD, Volume 5, Number 4, 2018). Improved and/or alternative treatments for hallucinations and/or dementia-related psychosis, which are effective, safe, and clinically well-tolerated, remain an important unmet medical need.

The dopamine receptor D1 subtype (D1) is the most abundant dopamine receptor in the central nervous system, and plays an important role in multiple CNS functions. For many years the modulation of dopamine signaling in dopaminergic CNS disorders has been attempted with direct acting D1 receptor agonists, but various D1 agonist agents have achieved very limited success as lack of efficacy, safety, tolerability, including notably unacceptable adverse effects, have limited the utility of such agents. In addition, D1 agonists have bell-shaped dose response curves on cognitive endpoints which complicates and confounds clinical use. Thus, prior attempts to develop clinically useful direct D1 receptor agonists have been largely unsuccessful due to receptor desensitization, poor ADME/PK properties, and dose limiting side effects such as hypotension. Direct acting dopamine therapies are also limited in effectiveness due in part to high dose associated cognition impairment, seizure risk, and tolerance development. Thus, there remains a significant unmet need for safe, effective, and clinically tolerable treatments of hallucinations and/or dementia-related psychosis.

Mevidalen is a dopamine D1 receptor Positive Allosteric Modulator (D1 PAM) and represents a potential first-in-class treatment for hallucinations and/or psychosis. Mevidalen (CAS Registry No. 1638667-79-4) can be described chemically as 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone, and can be structurally represented as:

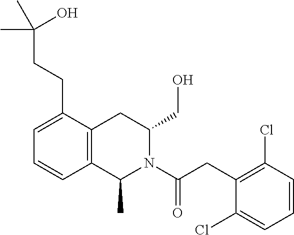 [SMILES: C[C@H]1c2cccc(CCC(C)(C)O)c2C[C@H](CO)N1C(=O)Cc1c(Cl)cccc1Cl]

Useful forms of mevidalen include a crystalline form (See WO 2017/070068), and a co-crystalline form comprising 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone and 4-hydroxybenzoic acid (CAS Registry No. 1638669-32-5) (See WO 2014/193781). As a positive allosteric modulator, also called a “potentiator” of the dopamine D1 receptor subtype, mevidalen is highly selective for D1. Mevidalen shows very weak direct agonism of the D1 receptor, and is active only in the presence of dopamine or a D1 agonist, and believed to be dependent on endogenous tone and subject to normal feedback control. Thus, mevidalen represents an innovative pharmacological agent and approach to modulating D1 signaling pathways for hallucinations and/or dementia-related psychosis where D1 signaling may be deficient. The underlying pathophysiology for hallucination's and psychosis symptoms are poorly understood, but involve activation of central dopamine, in some degree at D2 receptors, and serotonin receptors. Current therapies used to treat psychoses and hallucinations include atypical antipsychotic drugs which act through blockade of dopamine D2 receptors in the brain, and/or a serotonin 5-HT2A antagonist (see for example NUPLAZID® (pimavanserin) for L-DOPA induced psychosis/hallucinations in Parkinson's disease). However, NUPLAZID® can raise the risk of death in elderly people who have psychosis due to dementia, and is not currently approved for the treatment of patients with dementia-related psychosis unrelated to hallucinations and delusions associated with Parkinson's disease psychosis.

Mevidalen has a mechanism of action that differs from other dopaminergic agents such as the direct D1 receptor agonists. Mevidalen binds to a newly discovered allosteric binding site on intracellular loop 2 of the D1 receptor, where it increases the affinity of dopamine for the D1 receptor. Due to the complexity of dopaminergic signaling in normal physiology and clinical disease, and the lack of clinical pharmacological guidance from D1 orthosteric agonists, there remains an important unmet need for new methods of treatment using mevidalen and other dopamine D1 positive allosteric modulators. There remains an unmet need for methods of treatment for hallucinations and/or psychosis with dopamine D1 positive allosteric modulators, such as mevidalen, which provide a combined profile of effective, safe, and clinically tolerable pharmacological effects.

The D1PAM mevidalen was now been evaluated in a Phase 2 clinical study (referred to as PRESENCE (NCT03305809)) in patients to assess the safety and efficacy in patients with mild-to-moderate Lewy Body Dementia (Parkinson's Disease dementia—PDD or Dementia with Lewy Bodies—DLB) compared with placebo. This study had a primary endpoint for cognition and secondary endpoints including function, motor, sleep and other non-motor symptoms. Unexpectedly, it was discovered that mevidalen showed a surprising and improvement in hallucinations in these patients. In view of this discovery, the dopamine D1 specificity of mevidalen is hypothesized to be of importance for improved treatment of hallucinations and/or psychosis, in particular dementia-related psychosis. The present disclosure provides methods of using mevidalen, and/or pharmaceutical compositions thereof, and/or other dopamine D1 positive allosteric modulators, for use in the treatment of hallucinations and/or dementia-related psychosis.

In the methods of treatment of the present disclosure, and as used herein, mevidalen is 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone in any form, and includes crystalline and co-crystalline forms thereof, in particular the benzoic acid co-crystalline form, and/or pharmaceutical compositions comprising these agents. The present disclosure provides methods for use of mevidalen in the treatment of hallucinations and/or psychosis in a patient in need thereof, comprising administering to said patient a dose of about 5 mg to about 60 mg, up to a maximum total dose of 60 mg per day, of mevidalen, or pharmaceutical composition thereof. Preferably the psychosis is dementia-related psychosis. The present disclosure further provides a method for use of mevidalen in the treatment of hallucinations and/or dementia-related psychosis in a patient in need thereof, comprising administering to said patient a dose of about 10 mg to about 50 mg, up to a maximum total dose of 50 mg per day, of mevidalen, or a pharmaceutical composition thereof. Methods of treatment using mevidalen, and/or other D1 PAM agents described herein, provide a novel approach to prevent and/or relieve the symptoms of psychosis and/or hallucinations, without the side effects associated with dopamine D2 antagonists (such as atypical antipsychotics), and may be used as a stand alone therapy in neurodegenerative disorders, or as a combination therapy, for instance in schizophrenia and/or bipolar disorders.

Mevidalen, has been studied in a Phase 2 clinical study for Parkinson's Disease Dementia (PRESENCE, NCT03305809) and was discovered to have an advantageous ability to treat hallucinations and psychosis. The PRESENCE study, having a summary description provided in Example 1, has resulted in the discovery that mevidalen when used according to the methods and dosing regimens of the present disclosure may induce a surprising and marked improvement in signs and symptoms of hallucinations and/or dementia-related psychosis. Thus, when used according to the present methods of treatment and dosing regimens, mevidalen provides a means to improve dopamine D1 signaling in a manner that provides an effective, safe, and clinically tolerable therapeutic regimen for prevention and/or treatment of hallucinations and/or dementia-related psychosis.

The methods of treatment and dosing regimens of the present disclosure provide surprising and unpredictable advantages. Patients having hallucinations or dementia-related psychosis have a need to avoid substantial risk of agitation, and/or undesired cardiovascular effects, such as elevations in pulse and blood pressure, while at the same time benefiting from D1 PAM activities which prevent or treat hallucinations and/or dementia-related psychosis symptoms. Unexpectedly it has been discovered that clinically useful and desirable effects of mevidalen for prevention and/or treatment of hallucinations, and/or dementia-related psychosis, can in fact be separated from certain undesirable effects by using the methods and clinical dosing regimens of the present disclosure.

Approximately 1% of adults in the U.S. suffer from schizophrenia, a debilitating and lifelong condition. See, e.g., NAMI, Mental Help, PsyCom, SAMHSA study, NIMH data consolidation. According to the American Psychiatric Association, 30% of schizophrenia patients (approximately 700,000) have an inadequate response to their antipsychotic therapy. In this context antipsychotic therapies include for example quetiapine, clozapine, aripiprazole, asenapine, cariprazine, brexpiprazole, lurasidone, olanzapine, and risperidone, and/or long-acting formulations thereof, including long-acting aripiprazole or risperidone.

An aspect of the present disclosure relates to methods of preventing or treating hallucinations and/or psychosis in a patient suffering from schizophrenia, wherein the patient has had a partial but inadequate response to other antipsychotic treatments. In another aspect, the disclosure relates to a method of preventing, treating, or diminishing a psychotic symptom, in a patient having a partial or completely inadequate response to an antipsychotic alone, comprising administering an effective amount of mevidalen, or a D1 PAM described herein, to the patient in need thereof, and continuing administration of the initial antipsychotic treatment. In certain embodiments, the other antipsychotic to be administered in combination is selected independently from the group consisting of quetiapine, clozapine, aripiprazole, asenapine, cariprazine, brexpiprazole, lurasidone, olanzapine, and risperidone, and/or long-acting formulations thereof, including long acting aripiprazole or risperidone.

The present disclosure provides certain clinical dosing regimens for the daily administration of mevidalen such that the patient suffering from hallucinations or psychosis will have relief of the signs and symptoms while avoiding other D1 PAM effects that would preempt these clinical benefits. In addition, the present disclosure provides for the chronic daily administration of mevidalen such that patient suffering hallucinations or psychosis will further be able to employ either lower or higher doses of mevidalen within the regimens of the present disclosure, such that effective symptomatic relief is achieved for the individual patient while undesirable effects are avoided. Generally, the dosing regimens of the present disclosure provide the means for patients to benefit from D1 PAM activity, while avoiding certain undesired adverse cardiovascular activities that have been observed clinically and may represent on-target pharmacology for D1 PAMs as a class. Further, the dosing regimens of the present disclosure provide a means to treat patients suffering from hallucinations or psychosis while at the same time decreasing the risk of drug-drug interactions with Cyp3A4 inhibitors.

Accordingly, the present disclosure provides dosing regimens for oral daily administration of mevidalen to a patient having hallucinations or psychosis, and in particular dementia-related psychosis, using certain doses of mevidalen which are described in detail below.

In an embodiment, the present disclosure provides a method for use of mevidalen in the prevention or treatment of hallucinations or psychosis, and in particular dementia-related psychosis, in a patient in need thereof, comprising administering to said patient a dose of about 5 mg to about 60 mg, up to a maximum total dose of 60 mg per day, of mevidalen, or pharmaceutical composition thereof.

In an embodiment, the present disclosure provides a method for use of mevidalen in the prevention or treatment of hallucinations or psychosis, and in particular dementia-related psychosis, in a patient in need thereof, comprising administering to said patient a dose of about 5 mg to about 60 mg, up to a maximum total dose of 60 mg per day, of mevidalen, or pharmaceutical composition thereof.

In an embodiment, the present disclosure provides a dopamine D1 positive allosteric modulator, or a pharmaceutically acceptable salt or co-crystal thereof, for use in the prevention or treatment of hallucinations or psychosis.

In an embodiment, the present disclosure provides a dopamine D1 positive allosteric modulator, or a pharmaceutically acceptable salt or co-crystal thereof, for use in the prevention or treatment of hallucinations or psychosis, wherein the psychosis is a dementia-related psychosis.

In an embodiment, the present disclosure provides a dopamine D1 positive allosteric modulator, or a pharmaceutically acceptable salt or co-crystal thereof, for use in the prevention or treatment of hallucinations or psychosis, wherein the patients' hallucinations or psychosis have been refractory to two or more prior antipsychotic therapies.

In an embodiment, the present disclosure provides a dopamine D1 positive allosteric modulator, or a pharmaceutically acceptable salt or co-crystal thereof, for use according to in the prevention or treatment of dementia-related psychosis, wherein the patient's dementia-related psychosis has been refractory to two or more prior antipsychotic therapies.

In an embodiment, the present disclosure provides mevidalen, or a pharmaceutically acceptable co-crystal thereof, for use in the prevention or treatment of hallucinations or psychosis.

In an embodiment, the present disclosure provides mevidalen, or a pharmaceutically acceptable co-crystal thereof, for use in the prevention or treatment of hallucinations or psychosis, wherein the psychosis is a dementia-related psychosis.

In an embodiment, the present disclosure provides mevidalen, or a pharmaceutically acceptable co-crystal thereof, for use in the prevention or treatment of hallucinations or psychosis, wherein the patients' hallucinations or psychosis have been refractory to two or more prior antipsychotic therapies.

In an embodiment, the present disclosure provides mevidalen, or a pharmaceutically acceptable co-crystal thereof, for use according any of the embodiments above, wherein the mevidalen, or a pharmaceutically acceptable co-crystal thereof, is orally administered daily in a dose of 5 to 60 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 10 to 50 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose selected from the group consisting of 10 mg, 15 mg, 20 mg, 25 mg, 30 mg, 35 mg, 40 mg, 45 mg, and 50 mg, per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 50 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 45 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 40 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 35 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 30 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 25 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 20 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 15 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 10 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is administered simultaneously, separately, or sequentially in combination with an atypical antipsychotic.

In an embodiment, the present disclosure provides mevidalen, or a pharmaceutically acceptable co-crystal thereof, for use according any of the embodiments above, wherein the mevidalen, or a pharmaceutically acceptable co-crystal thereof, is administered in combination with an atypical antipsychotic selected from the group consisting of quetiapine, clozapine, aripiprazole, asenapine, cariprazine, brexpiprazole, lurasidone, olanzapine, risperidone, and/or long acting formulations thereof.

In an embodiment, the present disclosure provides a method of preventing or treating hallucinations or psychosis in a patient in need thereof, comprising administering to said patient a dopamine D1 positive allosteric modulator, or salt or co-crystal thereof.

In an embodiment, the present disclosure provides a method of preventing or treating hallucinations or psychosis in a patient in need thereof, comprising administering to said patient a dopamine D1 positive allosteric modulator, or salt or co-crystal thereof, wherein the psychosis is a dementia-related psychosis.

In an embodiment, the present disclosure provides a method of preventing or treating hallucinations or psychosis in a patient in need thereof, comprising administering to said patient a dopamine D1 positive allosteric modulator, or salt or co-crystal thereof, wherein the patients' hallucinations or psychosis have been refractory to two or more prior antipsychotic therapies.

In an embodiment, the present disclosure provides a method of preventing or treating dementia-related psychosis in a patient in need thereof, comprising administering to said patient a dopamine D1 positive allosteric modulator, or salt or co-crystal thereof, wherein the patients' dementia-related psychosis has been refractory to two or more prior antipsychotic therapies.

In an embodiment, the present disclosure provides a method of preventing or treating hallucinations or psychosis in a patient in need thereof, comprising administering to said patient mevidalen, or a pharmaceutically acceptable co-crystal thereof, wherein the psychosis is a dementia-related psychosis.

In an embodiment, the present disclosure provides a method of preventing or treating hallucinations or psychosis in a patient in need thereof, comprising administering to said patient mevidalen, or a pharmaceutically acceptable co-crystal thereof, wherein the patients' hallucinations or psychosis have been refractory to two or more prior antipsychotic therapies.

In an embodiment, the present disclosure provides a method of preventing or treating dementia-related psychosis in a patient in need thereof, comprising administering to said patient mevidalen, or a pharmaceutically acceptable co-crystal thereof, wherein the patients' dementia-related psychosis has been refractory to two or more prior antipsychotic therapies.

In an embodiment, the present disclosure provides a method according to any of the method embodiments above, wherein the mevidalen, or a pharmaceutically acceptable co-crystal thereof, is orally administered daily in a dose of 5 to 60 mg per dose.

In an embodiment, the present disclosure provides a method according to any of the method embodiments above, wherein the mevidalen, or a pharmaceutically acceptable co-crystal thereof, is orally administered daily in a dose of 10 to 50 mg per dose.

In an embodiment, the present disclosure provides a method according to any of the method embodiments above, wherein the mevidalen, or a pharmaceutically acceptable co-crystal thereof, is orally administered daily in a dose selected from the group consisting of 10 mg, 15 mg, 20 mg, 25 mg, 30 mg, 35 mg, 40 mg, 45 mg, and 50 mg, per dose.

In an embodiment, the present disclosure provides a method according to any of the method embodiments above, wherein the mevidalen, or a pharmaceutically acceptable co-crystal thereof, is orally administered daily in a dose of 50 mg per dose.

In an embodiment, the present disclosure provides a method according to any of the method embodiments above, wherein the mevidalen, or a pharmaceutically acceptable co-crystal thereof, is orally administered daily in a dose of 45 mg per dose.

In an embodiment, the present disclosure provides a method according to any of the method embodiments above, wherein the mevidalen, or a pharmaceutically acceptable co-crystal thereof, is orally administered daily in a dose of 40 mg per dose.

In an embodiment, the present disclosure provides a method according to any of the method embodiments above, wherein the mevidalen, or a pharmaceutically acceptable co-crystal thereof, is orally administered daily in a dose of 35 mg per dose.

In an embodiment, the present disclosure provides a method according to any of the method embodiments above, wherein the mevidalen, or a pharmaceutically acceptable co-crystal thereof, is orally administered daily in a dose of 30 mg per dose.

In an embodiment, the present disclosure provides a method according to any of the method embodiments above, wherein the mevidalen, or a pharmaceutically acceptable co-crystal thereof, is orally administered daily in a dose of 25 mg per dose.

In an embodiment, the present disclosure provides a method according to any of the method embodiments above, wherein the mevidalen, or a pharmaceutically acceptable co-crystal thereof, is orally administered daily in a dose of 20 mg per dose.

In an embodiment, the present disclosure provides a method according to any of the method embodiments above, wherein the mevidalen, or a pharmaceutically acceptable co-crystal thereof, is orally administered daily in a dose of 15 mg per dose.

In an embodiment, the present disclosure provides a method according to any one of the methods above, wherein the mevidalen, or a pharmaceutically acceptable co-crystal thereof, is orally administered daily in a dose of 10 mg per dose.

In an embodiment, the present disclosure provides a method of preventing or treating hallucinations or psychosis in a patient in need thereof, comprising administering to said patient a dopamine D1 positive allosteric modulator, or salt or co-crystal thereof, in combination with an atypical antipsychotic agent.

In an embodiment, the present disclosure provides a method of preventing or treating hallucinations or psychosis in a patient in need thereof, comprising administering to said patient a dopamine D1 positive allosteric modulator, or salt or co-crystal thereof, in combination with an atypical antipsychotic agent, wherein the atypical antipsychotic agent is selected from the group consisting of quetiapine, clozapine, aripiprazole, asenapine, cariprazine, brexpiprazole, lurasidone, olanzapine, risperidone, and/or long acting formulations thereof.

As used above, and throughout the description of the invention, the following terms, unless otherwise indicated, shall be understood to have the following meanings:

A “pharmaceutically acceptable carrier, diluent, or excipient” is a medium generally accepted in the art for the delivery of biologically active agents to mammals, e.g., humans.

A “dose” refers to a predetermined quantity or unit dose of mevidalen calculated to produce the desired therapeutic effect in a patient. As used herein “mg” refers to milligram. As used herein, dose ranges and doses provided of mevidalen represent the weight of the active pharmaceutical ingredient, 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone, regardless of the form in which it is provided, such as the free base, a cocrystalline form, or any other composition or form. Preferably unit doses are comprised of 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone and 4-hydroxybenzoic acid in cocrystalline form. The term “about” as used herein, means in reasonable vicinity of the stated numerical value, such as plus or minus 10% of the stated numerical value.

Methods of making and formulating mevidalen and/or 2-(2,6-dichlorophenyl)-1-((1S,3R)-5-(2-hydroxy-2-methylpropyl)-3-(hydroxymethyl)-1-methyl-3,4-dihydroisoquinolin-2(1H)-yl)ethan-1-one, are known in the art and recited for example in WO 2014/193781 and/or WO 2017/070068. Methods of preparing mevidalen and co-crystals thereof, and certain formulations and dosage forms thereof, are known to the skilled artisan, and are described in WO 2014193781 and/or WO 2017/070068. WO 2014/193781 discloses certain 3,4-dihydroisoquinolin-2(1H)-yl compounds as positive allosteric modulators (PAM) of the dopamine 1 receptor (D1), including 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone and a cocrystalline form comprising 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone and 4-hydroxybenzoic acid, and compositions thereof. WO 2017/070068 discloses crystalline 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone. Mevidalen is preferably formulated as pharmaceutical composition administered by any route which makes the compound bioavailable, including oral, intravenous, and transdermal routes. Most preferably, such pharmaceutical compositions are for oral administration. Mevidalen can be administered alone or in the form of a pharmaceutical composition with pharmaceutically acceptable carriers, diluents or excipients. Throughout the description, where compositions are described as having, including, or comprising specific components, it is contemplated that compositions also consist essentially of, or consist of, the recited components. Such pharmaceutical compositions and processes for making the same are known in the art (See, e.g., Remington: The Science and Practice of Pharmacy, L. V. Allen, Editor, 22nd Edition, Pharmaceutical Press, 2012). In a formulation mevidalen is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the formulations can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, gels, suppositories, sterile injectable solutions, and sterile packaged powders. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compounds of the invention can be formulated so as to provide quick, sustained or delayed release of the active ingredient after administration to the patient by employing procedures known in the art. One skilled in the art of preparing formulations can readily select the proper form and mode of administration depending upon the particular characteristics of the compound and/or form selected, the disorder or condition to be treated, the stage of the disorder or condition, and other relevant circumstances. The table below provides examples of selected unit dosage forms provided as tablets for oral administration according to the dosing regimens of the present invention. The skilled artisan can use these examples, along with readily known formulation methods, to provide additional formulations and/or unit dosage forms.

| | Unit Dose (mg mevidalen, active ingredient) | | | | |
|---|---|---|---|---|---|
| | 5 | 10 | 25 | 30 | 50 |
| Component | mg | mg | mg | mg | mg |
| Mevidalen - | 6.5 | 13.1 | 32.7 | 39.2 | 65.3 |
| Benzoic Acid | | | | | |
| Cocrystal (mg) | | | | | |
| Microcrystalline | 109.7 | 126.4 | 81.1 | 142.8 | 162.2 |
| Cellulose | | | | | |
| Croscarmellose | 6.3 | 7.5 | 6.3 | 10 | 12.5 |
| Sodium | | | | | |
| Sodium Stearyl | 2.5 | 3 | 2.5 | 4 | 5 |
| Fumarate | | | | | |
| Silixon Dioxide, | 0 | 0 | 2.5 | 4 | 5 |
| Core Tablet | 125 | 150 | 125 | 200 | 250 |
| Weight (mg): | | | | | |
| Coating: | 5 | 6 | 5 | 8 | 10 |

The unit doses of the present invention are formulated as pharmaceutical compositions administered by any route which makes the compound bioavailable, preferably such compositions are for oral administration. “Administration” or “administering”, as used herein, includes wherein the patient self-administers mevidalen, and/or wherein mevidalen is administered by another person, and/or wherein the patient is instructed and/or directed to consume mevidalen according to a particular regimen. Preferably mevidalen is administered in the morning. Preferably, mevidalen is taken daily. Preferably the indicated unit doses of mevidalen are taken daily, that is one time per day, as is indicated by the use of the term “per day”. As used herein, “daily administration” includes the administration of mevidalen as a specific treatment regimen intended to provide the beneficial effect from the long term and regular administration of mevidalen at the specified doses. In particular, “daily administration” includes administration every day consecutively for not less than twenty-one days in a row, or for as long as is needed to prevent the patients' signs and symptoms of a dopaminergic CNS disorder. If a patient misses an occasional day, then the patient may simply resume administration on the next day specified for administration, and such an instance would continue to represent “daily administration”. As used herein, “daily” means mevidalen is administered one time every 24-hour period, or one time every calendar day. As used herein, “daily” means mevidalen is administered on an ongoing consecutive basis, where administering includes as used herein includes both when the patient administers the doses, and/or wherein the patient is instructed to administer the doses as part of a treatment regimen. Where methods are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

Embodiments of the present disclosure include other dopamine D1 positive allosteric modulators, for instance as those described and/or exemplified in WO 2014193781 and/or WO 2017/070068, and WO 2019/204419, and WO 2016/055479, including any salts and/or co-crystals thereof. For instance, one such other dopamine D1 positive allosteric modulator is referred to as DPTQ, which is 2-(2,6-dichlorophenyl)-1-((1S,3R)-5-(2-hydroxy-2-methylpropyl)-3-(hydroxymethyl)-1-methyl-3,4-dihydroisoquinolin-2(1H)-yl)ethan-1-one, is shown below.

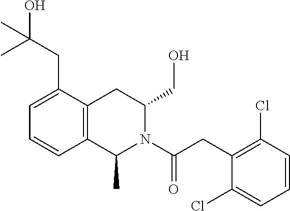 [SMILES: C[C@H]1c2cccc(CC(C)(C)O)c2C[C@H](CO)N1C(=O)Cc1c(Cl)cccc1Cl]

Embodiments of the present disclosure include other dopamine D1 positive allosteric modulators, for instance as those listed below, for instance a compound of the formula Ib:

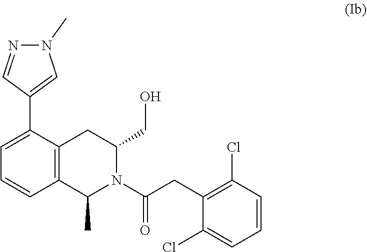 [SMILES: C[C@H]1c2cccc(-c3cnn(C)c3)c2C[C@H](CO)N1C(=O)Cc1c(Cl)cccc1Cl]

or a pharmaceutically acceptable salt or co-crystal thereof, which in the free base form can also be named as 2-(2,6-dichlorophenyl)-1-((1S,3R)-3-(hydroxymethyl)-1-methyl-5-(1-methyl-1H-pyrazol-4-yl)-3,4-dihydroisoquinolin-2(1H)-yl)ethan-1-one.

Embodiments of the present disclosure include other dopamine D1 positive allosteric modulators, for instance as those disclosed herein, or a pharmaceutically acceptable salt thereof or co-crystal thereof, and a pharmaceutically acceptable carrier, diluent or excipient.

Embodiments of the present disclosure include other dopamine D1 positive allosteric modulators, for instance as those listed below:

The present invention provides a compound which is 2-(2,6-dichlorophenyl)-1-((1S,3R)-5-(1-ethyl-1H-pyrazol-4-yl)-3-(hydroxymethyl)-1-methyl-3,4-dihydroisoquinolin-2(1H)-yl)ethan-1-one, or a pharmaceutically acceptable salt thereof.

The present invention provides a compound which is 1-((1S,3R)-5-(1-cyclopropyl-1H-pyrazol-4-yl)-3-(hydroxymethyl)-1-methyl-3,4-dihydroisoquinolin-2(1H)-yl)-2-(2,6-dichlorophenyl)ethan-1-one, or a pharmaceutically acceptable salt thereof.

The present invention provides a compound which is 2-(2,6-dichlorophenyl)-1-((1S,3R)-3-(hydroxymethyl)-1-methyl-5-(1-(oxetan-3-yl)-1H-pyrazol-4-yl)-3,4-dihydroisoquinolin-2(1H)-yl)ethan-1-one, or a pharmaceutically acceptable salt thereof.

The present invention provides a compound which is 1-((1S,3R)-5-(1-(2-(11-oxidaneyl)ethyl)-1H-pyrazol-4-yl)-3-(hydroxymethyl)-1-methyl-3,4-dihydroisoquinolin-2(1H)-yl)-2-(2-chlorophenyl)ethan-1-one, or a pharmaceutically acceptable salt thereof.

The present invention provides a compound which is 2-(2-chloro-6-fluorophenyl)-1-((1S,3R)-3-(hydroxymethyl)-1-methyl-5-(1-(oxetan-3-yl)-1H-pyrazol-4-yl)-3,4-dihydroisoquinolin-2(1H)-yl)ethan-1-one, or a pharmaceutically acceptable salt thereof.

The present invention provides a compound which is 2-(2-chlorophenyl)-1-((1S,3R)-3-(hydroxymethyl)-1-methyl-5-(1-methyl-1H-pyrazol-4-yl)-3,4-dihydroisoquinolin-2(1H)-yl)ethan-1-one, or a pharmaceutically acceptable salt thereof.

The present invention provides a compound which is 2-(2,6-dichlorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2-chloro-6-fluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2,6-difluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2-chloro-5-fluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2-chloro-4-fluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2-fluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2,3-difluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2,5-difluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

Embodiments of the present disclosure includes dopamine D1 positive allosteric modulators above and/or salt or co-crystal thereof, and a pharmaceutically acceptable carrier, diluent or excipient.

As used herein, the term “patient” refers to a human, and patients to be treated by the present dosing regimens are hallucinations and/or psychosis patients, and preferably dementia-related psychosis patients, and as such share etiopathological aspects in that disturbances of dopamine signaling contribute to these hallucinations and/or psychosis. Identification of patients with hallucinations and/or psychosis can be achieved by established methods known to the skilled artisan and described herein, for instance in Example 1.

In embodiments of the invention a patient is a human who has been diagnosed as having a medical risk, condition, or disorder, such as hallucinations, or dementia-related psychosis, in need of treatment with a D1 PAM, such as mevidalen, and using a dosing regimen described herein. In those instances where the disorders which can be prevented or treated by the methods of the present invention are known by established and accepted classifications, such as hallucinations and/or psychosis, their classifications can be found in various well-known medical texts. For example, at present, the 5th edition of the Diagnostic and Statistical Manual of Mental Disorders (DSM-5), provides a diagnostic tool for identifying many of the disorders described herein. Also, the International Classification of Diseases, Tenth Revision (ICD-10), provides classifications for many of the disorders described herein. The skilled artisan will recognize that there are alternative nomenclatures, nosologies, and classification systems for disorders described herein, including those as described in the DSM-5 and ICD-10, and that terminology and classification systems evolve with medical scientific progress. Hallucinations and delusions and psychosis are well described and known to the skilled artisan, and the diagnostic methods described herein and as well as in the medical literature are used to identify patients in need of the present methods of treatment and prevention. While dementia-related hallucinations, dementia-related delusions, and dementia-related psychosis patients are preferred patients of the present methods, patients of various other disease origins having hallucinations, delusions, and or psychosis are also patients in need of prevention and/or treatment according to the methods described herein. Diagnostic criteria in DSM-5 (5th edition of the Diagnostic and Statistical Manual of Mental Disorders) describe evidence of significant clinical and quality of life impact arising from hallucinations, delusions, and psychosis, and pathologies may or may not interfere with independence in everyday activities.

The APA and the World Health Organization define psychosis narrowly by requiring the presence of hallucinations (without insight into their pathologic nature), delusions, or both hallucinations without insight and delusions. In both current diagnostic classification systems, impaired reality testing remains central conceptually to psychosis. In contrast to earlier diagnostic classification systems, the current systems operationalize impaired reality testing by identifying the symptoms that provide evidence of such impairment. Delusions (i.e., fixed false beliefs), by definition, are evidence of impaired reality testing: delusional beliefs are ones maintained steadfastly even in the face of evidence contradicting them incontrovertibly.

Similarly, hallucinations (i.e., perceptions occurring in the absence of corresponding external or somatic stimuli) are evidence of impaired reality when the individual experiencing them is unable to recognize the hallucinatory nature of such experiences. Both the current APA and the World Health Organization classification systems acknowledge that “formal thought disorder” (ie, disorganized thinking, including illogicality, tangentiality, perseveration, neologism, thought blocking, derailment, or some combination of these disturbances of thought) is one of several commonly co-occurring features of psychotic disorders. The DSM-5 allows formal thought disorder to supplant hallucinations and delusions in the diagnosis of a psychotic disorder when it is accompanied by grossly disorganized behavior, catatonia (for schizophrenia, schizophreniform, brief psychotic, and schizoaffective disorders) and/or negative symptoms (for schizophrenia, schizophreniform, and schizoaffective disorders but not brief psychotic disorder), alone or in combination. Since mildly disorganized speech is common and diagnostically nonspecific, the degree of thought disorder required to fulfill this DSM-5 criterion must be of severity sufficient to substantially impair effective communication. As used herein, the term psychosis may refer to the presence of delusions, hallucinations without insight, or both. These symptoms are clearly defined common features of psychosis in both psychiatric disorders and neurologic conditions. They are captured by informal and structured clinical assessments (See Psychosis, David B. Arciniegas, Continuum (Minneap Minn) 2015; 21(3): 715-736).

Hallucination is defined as a sensory perception in the absence of a corresponding external or somatic stimulus and described according to the sensory domain in which it occurs. Hallucinations may occur with or without insight into their hallucinatory nature. The absence of insight into a hallucination defines it as a psychotic symptom, that is, a hallucination for which reality testing is impaired. Hallucinations without insight are contrasted with hallucinations that the individual recognizes as unreal. Examples of hallucinations with preserved insight include the visual hallucinations of migraine aura, sleep transition-related hypnagogic (while falling asleep) and hypnopompic (while waking) hallucinations, and the hallucinated hearing of one's name being called that many psychiatrically and neurologically healthy individuals experience occasionally.

The management of patients with hallucinations or psychosis is often unsatisfactory because available acute and preventive therapies are either ineffective or poorly tolerated. The acute treatment of hallucinations or psychosis has been limited to the use of atypical antipsychotics and 5HT-2A antagonists. Mevidalen represents an innovative approach for acute therapy or prevention of hallucinations or psychosis by selectively targeting and positively modulating dopamine D1 receptors. While some patients will be able to successfully manage hallucinations and psychosis by treatment with current antipsychotic agents alone, a population of patients will fail to successfully manage their hallucinations and psychosis with these agents. These patients may have a number of hallucinations per day or per-month that continues to be significantly disabling. Further, some patients, referred to herein as refractory patients, will fail to successfully manage their hallucinations and psychosis and will suffer from hallucinations and psychosis which are refractory to two or more prior antipsychotic prevention and/or treatment regimens. As defined herein, refractory hallucinations and/or psychosis patients, or refractory dementia-related psychosis patients, will be those that continue to suffer hallucinations or psychosis on 1 or more days per month, despite two or more prior prevention or treatment regimens. As used herein, prior prevention or treatment regimens means prior unsatisfactory treatment attempts with an antipsychotic treatment regimen, such as quetiapine, clozapine, aripiprazole, asenapine, cariprazine, brexpiprazole, lurasidone, olanzapine, risperidone, and/or long-acting formulations thereof, either alone or in combination. Refractory patients have yet to achieve substantial freedom from recurrent hallucinations or psychosis, and thus represent a critical unmet need. Failure of these refractory patients to achieve adequate relief from multiple prior treatment regimens demonstrates that their disease is particularly difficult to treat, and efficacy in this population represents a surprising and superior outcome.

The terms prevention or preventing as used herein refer to all processes wherein there may be a significant reduction, and/or more preferably freedom from significant signs and symptoms of hallucinations and or psychosis.

The terms “treatment” and “treating” are intended to refer to all processes wherein there may be a slowing, interrupting, arresting, controlling, or stopping of the progression of an existing disorder and/or symptoms thereof, but does not necessarily indicate a total elimination of all symptoms.

The attending diagnostician, as one skilled in the art, can readily determine the dose chosen from the dosing regimens provided herein by observing results obtained from treatment. In determining a specific dose of Mevidalen from dosing regimens of the present invention, a number of factors are considered, including, but not limited to the dopaminergic CNS disorder from which the patient suffers, the weight, age, and general health of the patient; the degree of involvement or the severity of the disorder; the response of the individual patient; the use of other concomitant medication; and other relevant circumstances.

A dose regimen of the present invention may be used in combination with other drugs that are used in the treatment/prevention/suppression or amelioration of hallucinations and/or dementia related psychosis. Such other drug(s) may be administered, by a route and in an amount commonly used therefore, contemporaneously, or sequentially with Mevidalen. For example, other active ingredients effective in the treatment of Parkinson's disease that may be combined with Mevidalen, include, but are not limited to atypical antipsychotics and 5HT-2A antagonists described herein.

### BRIEF DESCRIPTION OF THE DRAWINGS

FIG. 1: Mevidalen treatment resulted in significant improvements in hallucinations and psychosis. In the PRESENCE study, participants with LBD, aged 40-85 years, and with a Hoehn and Yahr score of 0-4 and a Montreal Cognitive Assessment score of 10-23 were randomized 1:1:1:1 to mevidalen 10 mg, 30 mg, or 75 mg or placebo once-daily for 12 weeks (See Example 1).

### EXAMPLE 1: PHASE 2 CLINICAL STUDY FOR PARKINSON'S DISEASE DEMENTIA (REFERRED TO AS PRESENCE, NCT03305809)

The objective of this study was to assess the motor and non-motor effects of mevidalen in patients with Lewy Body Dementia (LBD) as measured by the MDS-UPDRS. Mevidalen is a positive allosteric modulator of D1 receptor (D1PAM). D1 receptor activation improves cognitive and motor function and enhances wakefulness in preclinical and clinical models. The Phase 2, 12-week PRESENCE study was designed to evaluate the symptomatic effect of mevidalen for treatment of cognition and other domains relevant to LBD including motor function, sleep, mood and apathy.

Participants with LBD, aged 40-85 years, and with a Hoehn and Yahr score of 0-4 and a Montreal Cognitive Assessment score of 10-23 were randomized 1:1:1:1 to mevidalen 10 mg, 30 mg, or 75 mg or placebo once-daily. The primary cognition outcome was CDR-CoA. Secondary outcomes included change from baseline to Week 12 in the MDS-UPDRS total score (sum of Parts I-III) and change in both the MDS-UPDRS Part II (motor experiences of daily living) and Part III (motor exam) scores. Analyses were also pre-specified for the Part I items of fatigue, daytime sleepiness, hallucinations, depressed mood and apathy and the Part IV items of motor fluctuations and dyskinesias.

Below is provided a protocol for study of mevidalen in PD with certain doses of the methods of treatment and dosing regimens described herein. The skilled artisan will be able to apply the teachings of this Example 1, and other disclosures provided herein, and conduct similar studies with additional doses and dosing regimens of the present invention.

Treatments for hallucinations and/or dementia related psychosis, that is well tolerated, remains an unmet medical need. Mevidalen has for the first time as described herein been observed to improve hallucinations and/or dementia related psychosis in a relatively short period of chronic daily dosing (12 weeks). As a symptomatic agent, mevidalen is important for patients who continue to suffer from hallucinations and/or dementia-related psychosis.

The PRESENCE study assessed three doses (10 mg, 30 mg, and/or 75 mg daily (or 50 mg based on interim analysis) (QD) oral dosing) of mevidalen vs. placebo over 12 weeks of treatment. The primary outcome was a measure of cognition and but also assessed additional endpoints described herein. PRESENCE was a randomized placebo-controlled trial in individuals with Parkinson's disease dementia to evaluate the safety and efficacy of (three doses of study drug) mevidalen in participants with mild-to-moderate Parkinson's disease dementia treated for 12 weeks.

Study HBEH included subjects who meet the revised MDS criteria for PD (Postuma et al. 2015) and mild-to-moderate dementia as defined by a decline in cognitive function, which in the opinion of the investigator has resulted in functional impairment and a MoCA score between 10 and 23 (Trzepacz et al. 2015). Per the revised MDS criteria, PDD can be diagnosed in the presence of dementia, regardless of the timing of dementia onset relative to PD diagnosis. Subjects diagnosed with dementia with Lewy bodies (DLBs) should be considered as also having PD if they meet the MDS PD criteria. Therefore, subjects may have dementia prior to, at the time of, or subsequent to the diagnosis of PD. Unlike registration trials of symptomatic therapies in PDD (Emre et al. 2004), the current study may include some subjects who would otherwise have met the traditional criteria (dementia prior to or within 1 year of motor onset) for DLB based on the timing of their dementia (Mckeith et al. 2005). This criterion was that the dementia occurs prior to or within 1 year of Parkinson's symptoms. The 1-year rule is arbitrary and based on the historical belief that PD was not associated with dementia; however, there is increasing controversy about the validity of this traditional approach to splitting the diagnoses (Berg et al. 2014). In support of the proposed approach, both disorders share a variety of clinical, genetic, and pathological features (Lippa et al. 2007; Postuma et al. 2009; Johansen et al. 2010). Both DLB and PDD are associated with similar impairments in cognition with predominant visuoperceptual abnormalities, improvement in memory with cues, and so on. Both are associated with prominent psychosis, neuroleptic sensitivity, and alterations in arousal. Prodromal features (e.g., rapid eye movement [REM] sleep behavior disorder, olfactory loss) are the same in both conditions. Non-motor symptoms with depression, anxiety, autonomic dysfunction and sleep disturbances occur with similar relative frequency in both. The same genetic mutations (alpha-synuclein duplications, glucocerebrosidase mutations) are associated with the development of either condition. Finally, they have a shared pathology with alpha-synuclein and Lewy body formation in the brain stem and cortex. Therefore, the Study HBEH meets current thinking about PDD and DLB that, apart from the timing of cognitive impairment, they are clinically and pathologically indistinguishable and would likely respond to similar therapeutic approaches (Aarsland et al. 2004; Ballard et al. 2006). Placebo is included as the control, in a blinded manner for investigator and site staff and subjects, to allow for an unbiased assessment of the safety data generated, which will allow for a more robust comparison between mevidalen and placebo data. Comparison of 3 dosage levels of mevidalen was chosen to evaluate dosage exposure response for safety and efficacy. Initial visits (Visit 3 to Visit 7) were selected to occur at a weekly interval to provide a detailed evaluation of the efficacy and safety of mevidalen during the initial treatment A dosing duration of 12 weeks was selected, as it is estimated to be the minimum duration where a beneficial effect on cognition may be observed.

Primary objective was to test the hypothesis that mevidalen administered at 10 mg, 30 mg, and/or 75 mg daily (or 50 mg based on interim analysis) (QD) oral dosing for 12 weeks would result in a significant improvement in cognition in subjects with mild-to-moderate PDD compared with placebo. Primary endpoints are changes in the CoA composite score of the CDR-CCB from baseline to Week 12. Secondary objectives are described below.

| Secondary efficacy objectives | Secondary efficacy endpoints |
|---|---|
| To evaluate the global efficacy of | ADCS-CGIC score from baseline to |
| mevidalen | Week 12 |
| To evaluate the efficacy of mevidalen on | Change in the CDR-CCB PoA composite |
| cognitive outcomes | score from baseline to Week 12 |
| To evaluate the efficacy of mevidalen on | Change in the ADAS-Cog13 score from |
| cognitive outcomes | baseline to Week 12 |
| To evaluate the efficacy of mevidalen on | Change in the MoCA score from |
| cognitive outcomes | screening to Week 12 |
| To evaluate the efficacy of mevidalen on | Change in the NPI total and individual |
| neuropsychiatric symptoms | items scores from baseline to Week 12 |
| To evaluate the effect of mevidalen on | Change in the ESS score from baseline to |
| daytime sleepiness | Week 12 |
| To evaluate the effect of mevidalen on PD | Change in the MDS-UPDRS total score (sum |
| severity | of Parts I-III) from baseline to Week 12 |
| To evaluate the efficacy of mevidalen on | Change in the PDAQ-15 total score from |
| functional outcome | baseline to Week 12 |
| To evaluate the effect of mevidalen on | Change in D-KEFS Verbal Fluency test |
| verbal fluency | score from baseline to Week 12 |
| Safety objectives | Safety endpoints |
| To evaluate the effect of mevidalen on | Number of subjects who met the |
| acute changes of vital signs on the first | potentially clinically significant vital signs |
| day of dosing | criteria at 3 consecutive time points at |
| | Visit 3 (Day 1 stopping rules) |
| To evaluate the effect of mevidalen on | Change in in-clinic SBP from 0 up to 8 |
| SBP on the first day of dosing | hours post dose on the first day of study |
| | drug dosing |
| To evaluate the effect of mevidalen on | Change in in-clinic pulse rate from 0 up to |
| pulse rate on the first day of dosing | 8 hours post dose on the first day of study |
| | drug dosing |
| To evaluate the effect of mevidalen on | Change in in-clinic mean SBP at baseline |
| SBP from baseline to Week 12 | to mean SBP at Week 12 |
| To evaluate the effect of mevidalen on | Change in in-clinic mean pulse rate at |
| pulse rate from baseline to Week 12 | baseline to mean pulse rate at Week 12 |
| Pharmacokinetics objectives: | Pharmacokinetics objectives: |
| To assess the PK of mevidalen in a | Steady-state trough plasma concentrations |
| population of subjects with mild-to- | of mevidalen at Week 12 |
| moderate dementia due to PD | |

Abbreviations: ADAS-Cog13 = 13-item Alzheimer's Disease Assessment Scale - Cognitive subscale; ADCS-CGIC = Alzheimer's Disease Cooperative Study - Clinician Global Impression of Change; CDR-CCB = Cognitive Drug Research - Computerized Cognition Battery; CoA = Continuity of Attention; D-KEFS = Delis-Kaplan Executive Function System; ESS = Epworth Sleepiness Scale; MDS-UPDRS = Movement Disorder Society's Unified Parkinson's Disease Rating Scale; MoCA = Montreal Cognitive Assessment; NPI = Neuropsychiatric Inventory; PD = Parkinson's disease; PDD = Parkinson's disease dementia; PDAQ-15 = Penn Parkinson's Daily Activities Questionnaire-15; PK = pharmacokinetics; PoA = Power of Attention; QD = once a day; SBP = systolic blood pressure.

### REFERENCES

Postuma R B, Berg D, Stern M, Poewe W, Olanow C W, Oertel W, Obeso J, Marek K, Litvan I, Lang A E, Halliday G, Goetz C G, Gasser T, Dubois B, Chan P, Bloem B R, Adler C H, Deuschl G. MDS clinical diagnostic criteria for Parkinson's disease. Mov Disord. 2015; 30(12):1591-1601.
Trzepacz P T, Hochstetler H, Wang S, Walker B, Saykin A J; Alzheimer's Disease Neuroimaging Initiative. Relationship between the Montreal Cognitive Assessment and Mini-mental State Examination for assessment of mild cognitive impairment in older adults. BMC Geriatr. 2015; 15:107.
Emre M, Aarsland D, Albanese A, Byrne E J, Deuschl G, De Deyn P P, Durif F, Kulisevsky J, van Laar T, Lees A, Poewe W, Robillard A, Rosa M M, Wolters E, Quarg P, Tekin S, Lane R. Rivastigmine for dementia associated with Parkinson's disease. N Engl J Med. 2004; 351(24):2509-2518.
McKeith I G, Dickson D W, Lowe J, Emre M, O'Brien J T, Feldman H, Cummings J, Duda J E, Lippa C, Perry E K, Aarsland D, Arai H, Ballard C G, Boeve B, Burn D J, Costa D, Del Ser T, Dubois B, Galasko D, Gauthier S, Goetz C G, Gomez-Tortosa E, Halliday G, Hansen L A, Hardy J, Iwatsubo T, Kalaria R N, Kaufer D, Kenny R A, Korczyn A, Kosaka K, Lee V M, Lees A, Litvan I, Londos E, Lopez O L, Minoshima S, Mizuno Y, Molina J A, Mukaetova-Ladinska E B, Pasquier F, Perry R H, Schulz J B, Trojanowski J Q, Yamada M; Consortium on DLB. Diagnosis and management of dementia with Lewy bodies: third report of the DLB Consortium. Neurology. 2005; 65(12):1863-1872.
Berg D, Postuma R B, Bloem B, Chan P, Dubois B, Gasser T, Goetz C G, Halliday G M, Hardy J, Lang A E, Litvan I, Marek K, Obeso J, Oertel W, Olanow C W, Poewe W, Stern M, Deuschl G. Time to redefine PD? Introductory statement of the MDS Task Force on the definition of Parkinson's disease. Mov Disord. 2014; 29(4):454-462.
Lippa C F, Duda J E, Grossman M, Hurtig H I, Aarsland D, Boeve B F, Brooks D J, Dickson D W, Dubois B, Emre M, Fahn S, Farmer J M, Galasko D, Galvin J E, Goetz C G, Growdon J H, Gwinn-Hardy K A, Hardy J, Heutink P, Iwatsubo T, Kosaka K, Lee V M, Leverenz J B, Masliah E, McKeith I G, Nussbaum R L, Olanow C W, Ravina B M, Singleton A B, Tanner C M, Trojanowski J Q, Wszolek Z K; DLB/PDD Working Group. DLB and PDD boundary issues: diagnosis, treatment, molecular pathology, and biomarkers. Neurology. 2007; 68(11):812-819.
Postuma R B, Gagnon J F, Vendette M, Montplaisir J Y. Idiopathic REM sleep behavior disorder in the transition to degenerative disease. Mov Disord. 2009; 24(15):2225-2232.
Johansen K K, White L R, Sando S B, Aasly J O. Biomarkers: Parkinson disease with dementia and dementia with Lewy bodies. Parkinsonism Relat Disord. 2010; 16(5):307-315.
Aarsland D, Ballard C G, Halliday G. Are Parkinson's disease with dementia and dementia with Lewy bodies the same entity? J Geriatr Psychiatry Neurol. 2004; 17(3):137-145.
American Psychiatric Association. Diagnostic and statistical manual of mental disorders. 5th ed. Washington, D C; 2013.
Ballard C, Ziabreva I, Perry R, Larsen J P, O'Brien J, McKeith I, Perry E, Aarsland D. Differences in neuropathologic characteristics across the Lewy body dementia spectrum. Neurology. 2006; 67(11):1931-1934.
Yesavage J A, Brink T L, Rose T L, Lum O, Huang V, Adey M., Leirer V O. Development and validation of a geriatric depression screening scale: A preliminary report. J Psychiatr Res. 1983; 17:37-49.
Sheikh J I, Yesavage J A. Geriatric Depression Scale (GDS): Recent evidence and development of a shorter version. Clin Gerontologist. 1986; 5:165-173.

Summary of Study Design: Study 17S-MC-HBEH (HBEH) is a multicenter, randomized, double-blind, parallel-group, placebo-controlled, fixed-dosage, Phase 2a study comparing 3 dosages of mevidalen (10, or 30, or 75 mg administered orally [or 50 mg based on interim analysis] once a day [QD]) with placebo over 12 weeks in subjects with mild-to-moderate PDD. The study includes a Screening Period (Visits 1 to 2) of a minimum of 7 days and up to 14 days, a Pretreatment Period of a minimum of 11 days and up to 17 days (Visits 2 to 3), a 12-week Treatment Period (Visits 3 to 11), and a 14-day Safety Follow-Up Period (Visits 11 to 801 or early termination [ET]/discontinuation [DC] visit to Visit 801). Subjects who meet entry criteria will be randomized in a 1:1:1:1 ratio to mevidalen (10 or 30 or 75 mg QD) or placebo. The primary objective of the study is to test the hypothesis that mevidalen administration for 12 weeks will result in a significant improvement in cognition as measured by the change from baseline to Week 12 in the Continuity of Attention (CoA) composite score of the Cognitive Drug Research Computerized Cognition Battery (CDR-CCB), in subjects with mild-to-moderate PDD, compared to placebo. The CoA has demonstrated a significant treatment effect in previous trials in subjects with PDD (Wesnes et al. 2005; Rowan et al. 2007).

Treatment Arms and Duration: Study HBEH involves a comparison of mevidalen 10 mg, 30 mg, and 75 mg (or 50 mg based on interim analysis) administered orally QD with placebo over 12 weeks. Number of subjects: Approximately 400 subjects will be screened to achieve 340 randomized and an estimated total of 85 evaluable subjects per treatment group.

### Statistical Analysis:

Efficacy Analysis: All subjects in the evaluable patient population (EPP) will be considered for the efficacy analysis. The primary analysis on CoA will occur when all subjects complete 12 weeks of treatment. The analysis of CoA will utilize a Bayesian M MRM model. The Bayesian analysis may use uninformative priors for all terms in the model. These will be diffuse Normal distributions centered on zero. Priors for variance will follow an inverse gamma distribution. Further details of the Bayesian analysis will be provided in the SAP. The MMRM model will account for longitudinal data assessed throughout the study, after 1, 2, 4, 6, 8, 10, and 12 weeks of dosing. The change of CoA from the baseline to Week 12 will be the dependent variable. The model will comprise fixed (baseline value, treatment, visit) and random effects (subject) and the interaction terms (treatment by visit, baseline value by visit). Unstructured variance structure will be applied in the model, but if it fails to converge, other suitable structures will be investigated. The primary comparison will be the contrast (difference in least squares mean) between treatments and placebo for the Week 12 change from baseline. The secondary efficacy outcomes: the change from baseline at 12-week time point of total scores (or composite values) of Alzheimer's Disease Cooperative Study—Clinician Global Impression of Change (ADCS-CGIC), CDR-CCB Power of Attention (PoA), 13-item Alzheimer's Disease Assessment Scale—Cognitive subscale (ADAS-Cog13), Montreal Cognitive Assessment (MoCA), Neuropsychiatric Inventory (NPI), Epworth Sleepiness Scale (ESS), Movement Disorder Society's Unified Parkinson's Disease Rating Scale (MDS-UPDRS), Penn Parkinson's Daily Activities Questionnaire 15 (PDAQ-15), and Delis-Kaplan Executive Function System (D-KEFS) will follow the same analysis method as above. Missing records in some scales (e.g., ADAS-Cog) will be imputed as detailed in the statistical analysis plan. For the scales where the imputation is not done, if any item is missing, any total or sum involving that item will be considered missing. No adjustment for multiple comparisons will be made.

Safety Analysis: Safety analyses are based on the safety population and analysis will include listings and/or summaries of the following: adverse events (AEs), serious adverse events (SAEs), treatment-emergent adverse events (TEAEs), laboratory measures, vital signs, electrocardiogram readings and number of subjects who met the potentially clinically significant vital signs criteria at 3 consecutive time points at Visit 3 (Day 1 stopping rules). Mixed-model repeated measures analysis will be used to compare the change in in-clinic blood pressure (BP) and pulse rate from pretreatment up to 8 hours post dose measured on the first day of study drug dosing (V3). Two baselines will be considered in the change from baseline analyses: the V3 pretreatment value and the time-matched baselines from Visit 2 (hourly value 0 to 6 hours). For the second baseline, the V3, 7- and 8-hour time points will use the V2 6-hour time point as their baseline value. A separate change from baseline analysis will be completed for each baseline approach. Mixed-model repeated measures analyses will also be used to compare change in in-clinic BP and pulse rate from V2 (daily average 0 to 6 hours) to Week 6/Visit 8 and Week 12/Visit 11 (daily average 0 to 6 hours), to evaluate the change in BP and pulse rate over 12 weeks of dosing.

Pharmacokinetics (PK): Pharmacokinetic analyses will be conducted on subjects who receive at least 1 dose of the study drug and have 1 measurable concentration. A model-based approach may be implemented using nonlinear mixed effects modeling (NONMEM) or other appropriate software to estimate PK parameters.

Additional endpoints and biomarker data collected during the study may be evaluated in an exploratory manner.

Interim Analysis: Safety interim analyses will be conducted on the number of subjects on each treatment who met the potentially clinically significant vital signs criteria at 3 consecutive time points at Visit 3 (Day 1 stopping rules). This will be done after 50, 100, and 150 subjects have completed Visit 3. If there is >60% probability that the difference in rate of subjects meeting Day 1 stopping rules for 75 mg mevidalen compared to placebo is >0.3, the 75-mg dose level will be replaced with 50 mg for the subsequently enrolled subjects. Those already on 75-mg dose and passed the Day 1 stopping rules will remain on 75 mg. In the event of an unacceptable rate of subjects meeting Day 1 stopping rules at other doses, adjustments to doses may be made for subsequently randomized subjects at the discretion of the Internal Assessment Committee (IAC). Additional efficacy analyses may be conducted at the time of these safety interim analyses. A safety and efficacy interim analysis will be conducted when 170 randomized subjects have completed Visit 11 (Week 12) assessments. All potential efficacy analyses may be used for internal decision making, but are not planned to stop the study.

Study HBEH will include men and women aged 40 to 85 years with mild-to-moderate PDD. Subjects are eligible to be included in the study only if they meet all the following criteria at enrollment (Visit 1) (note that inclusion criteria [6] to [10] must be met or at an additional visit[s]):

Type of Subject and Disease Characteristics: [1] Male and female subjects aged 40 to 85 years (inclusive). [2] Have idiopathic PD per MDS criteria (Postuma et al. 2015) with at least 2 years of PD symptoms. [3] Have dementia as defined by a decline in cognitive function, which in the opinion of the investigator has resulted in functional impairment. [4] Have a MoCA score of 10 to 23 at the time of screening. [5] Are Modified Hoehn and Yahr Stages 1 to 4. [6] Have a BP or pulse rate at Visit 1 and Visit 3, as determined by 3 sequential BP/pulse rate measurements in the seated position:

For Subjects<60 years old: a mean systolic blood pressure (SBP) less than or equal to 140 mmHg, a mean diastolic BP less than or equal to 90 mmHg, and a mean pulse rate less than or equal to 90 beats/min in the seated position, and each of the 3 SBP measurement must be less than 180 mmHg.

For Subjects≥60 years old: a mean SBP less than or equal to 150 mmHg, a mean diastolic BP less than or equal to 90 mmHg, and a mean pulse rate less than or equal to 90 beats/min in the seated position, and each of the 3 SBP measurement must be less than 180 mmHg.

The following PD severity and cognitive assessments, as well as the Columbia-Suicide Severity Rating Scale (C-SSRS), will be done at Visit 1 as part of the subject eligibility evaluation: Movement Disorder Society (MDS) Clinical Diagnostic Criteria for Parkinson's disease Enrolled individuals will meet MDS criteria for clinically probable PD (Postuma et al. 2015). Subjects must have bradykinesia with either rest tremor and/or rigidity. Subjects must not have any absolute exclusion criteria described in Appendix 5. Subjects must not have the presence of greater than 2 red flags; if 1 red flag is present then it must be offset by 1 supportive criterion and if 2 red flags are present it must be offset by 2 supportive criteria.

In addition to meeting criteria for PD, subjects must meet criteria for dementia as described below (Montreal Cognitive Assessment [MoCA] Scale). The MDS criteria do not consider dementia as an exclusion criterion for PD and therefore there will be no restriction on the timing of dementia relative to the development of the motor features of PD.

Modified Hoehn and Yahr Scale: Enrolled individuals must be Hoehn and Yahr Stage 1 to Stage 4 at screening. The Hoehn and Yahr Scale (Hoehn and Yahr 1967) is used to describe the symptom progression of PD. The scale was originally described in 1967 and included Stages 1 through 5. It has since been modified with the addition of Stages 1.5 and 2.5 to account for the intermediate course of PD. The modified Hoehn and Yahr scale is as follows: Stage 0: No signs of disease, Stage 1: Unilateral disease, Stage 1.5: Unilateral plus axial involvement, Stage 2: Bilateral disease, without impairment of balance, Stage 2.5: Mild bilateral disease, with recovery on pull test, Stage 3: Mild-to-moderate bilateral disease; some postural instability; physically, independent, Stage 4: Severe disability; still able to walk or stand unassisted, Stage 5: Wheelchair bound or bedridden unless aided.

Montreal Cognitive Assessment Scale: Enrolled individuals must have a MoCA score of 10 to 23 at screening.

Geriatric Depression Scale: Enrolled individuals must have a Geriatric Depression Scale—Short Form (GDS-S) score of ≤6 at screening. The GDS is a site-administered questionnaire of depression in older adults (Yesavage et al. 1983). Users respond in a “Yes/No” format. Originally developed as a 30-item scale (Long Form), it has since been shortened to a 15-item scale (Short Form), which can be completed in approximately 5 to 7 minutes (Sheikh and Yesavage 1986). Of the 15 items, 10 are indicative of depression when answered “Yes” and 5 are indicative of depression when answered “No.” Columbia-Suicide Severity Rating Scale—Children's Version: The C-SSRS is a scale that captures the occurrence, severity, and frequency of suicide-related thoughts and behaviors during the corresponding assessment period. The C-SSRS, included here as a screening assessment, is described in detail in Section 9.4.4. The C-SSRS “Baseline” version will be used at screening, and the findings will constitute the baseline assessment. The C-SSRS will be administered to the subject after the cognitive and functional assessments. Responses from subject will be considered when administering the scale. If it is determined that the subject has suicidal ideation or behavior at this baseline assessment, then the subject will not be randomized and will be discontinued from the study.

### Table 1: Dose Dependent Effect of Mevidalen on Hallucinations and Psychosis Observed in PRESENCE.

The table below provides MDS-UPDRS Part I, Item Level data, presented as LSMean Change Difference from Baseline to Week 12, with P-value (treatment difference vs. placebo), and Effect size=treatment difference from LS Mean Difference/SD from VAR-COV matrix at Week 12.

| 10 mg mevidalen | | | 30 mg mevidalen | | | 75 mg mevidalen | | |
|---|---|---|---|---|---|---|---|---|
| LSMean | | | LSMean | | | LSMean | | |
| Change | P- | Effect | Change | P- | Effect | Change | P- | Effect |
| Difference | value | Size | Difference | value | Size | Difference | value | Size |
| −0.15 | 0.208 | −0.221 | −0.23 | 0.058 | −0.348 | −0.4 | 0.002 | −0.6 |

The PRESENCE trial also provided evidence that a cardiovascular effect is observed reflecting an acute increase in blood pressure and heart rate, and accommodation of these effects at lower doses of 10 and 30 mg. However, the adverse event profile and cardiovascular effects at the 75 mg dose may limit the clinical utility of higher doses. There, a preferred dosing regimen of the present invention is 10 mg to 50 mg daily for the prevention and/or treatment of hallucinations and/or dementia-related psychosis.

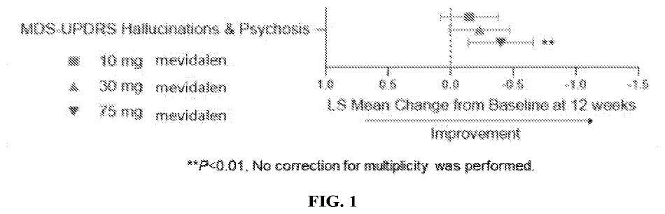

The data presented in Table 1 above provide evidence of therapeutic effect which prevents and/or reduces hallucinations and/or psychosis following daily administration of mevidalen at the indicated doses. See also FIG. 1. The effect represents a surprising and specific response in that other endpoints studied did not show similar dose dependent responses (for example MDS-UPDRS Part I, Item Level data for Apathy, Anxious Mood, and Depressed Mood measures did not show dose dependent therapeutic effect). Administration of mevidalen as described herein provides a novel approach to treat hallucinations and/or dementia-related psychosis arising in a variety of pathological conditions, including neurodegenerative disorders such as Parkinson's disease dementia (PDD), dementia with Lewy bodies (DLB), Alzheimer's disease (AD), Vascular dementia (VaD), and frontotemporal dementia (FTD). Clinical data and a substantial body of research suggest that psychotic symptoms can manifest independent of the underlying dementia subtype. The present methods of treatment using mevidalen are conceived to treat hallucinations and/or psychosis arising in a range of neuropsychiatric disorders, and dementia disorders in particular. The methods described herein are particularly useful for patients having the aforementioned neurodegenerative conditions, wherein the patient has further been diagnosed as experiencing hallucinations and or psychosis. Improvement in neuropsychiatric signs and symptoms in neurodegenerative diseases, as described herein for hallucinations and psychosis, is expected to provide a variety of possible real world clinical benefits for care of the affected patients, including decreased caregiver burden, increased quality of life, and potentially delayed progression to nursing home care or severe dementia.

1-19. (canceled)

20. A method of preventing or treating hallucinations or psychosis in a patient in need thereof, comprising administering to said patient a dopamine D1 positive allosteric modulator, or salt or co-crystal thereof.

21. A method according to claim 20, wherein the psychosis is a dementia-related psychosis.

22. A method according to claim 20, wherein the patients' hallucinations or psychosis have been refractory to two or more prior antipsychotic therapies.

23. A method according to any of claim 21 wherein the patients' dementia-related psychosis has been refractory to two or more prior antipsychotic therapies.

24. A method according to claim 20, wherein the D1 positive allosteric modulator is mevidalen, or a pharmaceutically acceptable co-crystal thereof.

25. A method according to claim 24, wherein the mevidalen, or a pharmaceutically acceptable co-crystal thereof, is orally administered daily in a dose of 5 to 60 mg per dose.

26. A method according to claim 24, wherein the mevidalen, or a pharmaceutically acceptable co-crystal thereof, is orally administered daily in a dose of 10 to 50 mg per dose.

27. A method according to claim 26, wherein the mevidalen, or a pharmaceutically acceptable co-crystal thereof, is orally administered daily in a dose selected from the group consisting of 10 mg, 15 mg, 20 mg, 25 mg, 30 mg, 35 mg, 40 mg, 45 mg, and 50 mg, per dose.

28. A method according to claim 26, wherein the mevidalen, or a pharmaceutically acceptable co-crystal thereof, is orally administered daily in a dose of 50 mg per dose.

29. A method according to claim 26, wherein the mevidalen, or a pharmaceutically acceptable co-crystal thereof, is orally administered daily in a dose of 45 mg per dose.

30. A method according to claim 26, wherein the mevidalen, or a pharmaceutically acceptable co-crystal thereof, is orally administered daily in a dose of 40 mg per dose.

31. A method according to claim 26, wherein the mevidalen, or a pharmaceutically acceptable co-crystal thereof, is orally administered daily in a dose of 35 mg per dose.

32. A method according to claim 26, wherein the mevidalen, or a pharmaceutically acceptable co-crystal thereof, is orally administered daily in a dose of 30 mg per dose.

33. A method according to claim 26, wherein the mevidalen, or a pharmaceutically acceptable co-crystal thereof, is orally administered daily in a dose of 25 mg per dose.

34. A method according to claim 26, wherein the mevidalen, or a pharmaceutically acceptable co-crystal thereof, is orally administered daily in a dose of 20 mg per dose.

35. A method according to claim 26, wherein the mevidalen, or a pharmaceutically acceptable co-crystal thereof, is orally administered daily in a dose of 15 mg per dose.

36. A method according to claim 26, wherein the mevidalen, or a pharmaceutically acceptable co-crystal thereof, is orally administered daily in a dose of 10 mg per dose.

37. A method of preventing or treating hallucinations or psychosis in a patient in need thereof, comprising administering simultaneously, separately, or sequentially, to said patient a dopamine D1 positive allosteric modulator, or salt or co-crystal thereof, in combination with an atypical antipsychotic agent.

38. A method according to claim 37 wherein the atypical antipsychotic agent is selected from the group consisting of quetiapine, clozapine, aripiprazole, asenapine, cariprazine, brexpiprazole, lurasidone, olanzapine, risperidone, and/or long-acting formulations thereof.
